(19) United States
(12) Patent Application Publication
(10) Pub. No.: US 2018/0271923 A1
(43) Pub. Date: Sep. 27, 2018

(54) COMPOSITION FOR PREVENTING OR TREATING MENOPAUSAL DISORDERS, CONTAINING TETRAGONIA TETRAGONOIDES (PALL.) KUNTZE EXTRACT

(71) Applicant: Korea Institute of Oriental Medicine, Daejeon (KR)

(72) Inventors: Byoung Seob Ko, Seoul (KR); Sun Min Park, Asan, Chungcheongnam-do (KR); Hye Won Lee, Daejeon (KR); Jin Ah Ryuk, Daejeon (KR); In Sil Park, Daejeon (KR); Su Ran Kim, Daejeon (KR)

(21) Appl. No.: 15/974,324

(22) PCT Filed: May 31, 2016
(86) PCT No.: PCT/KR2016/005755

§ 371 Date: May 8, 2018

(30) Foreign Application Priority Data
Nov. 10, 2015 (KR) 10-2015-0157664

(51) Int. Cl. A61K 36/185; A23L 33/105

(52) U.S. Cl. CPC A61K 36/185; A23V 2002/00; A23L 33/105

(57) ABSTRACT

The present invention relates to: a pharmaceutical composition for preventing or treating menopausal disorders, containing a Tetragonia tetragonoides (Pall.) Kuntze extract or a fraction thereof; a method for preventing or treating menopausal disorders, comprising a step of administering the pharmaceutical composition; and a food composition containing the extract or a fraction thereof. The Tetragonia tetragonoides (Pall.) Kuntze extract of the present invention shows effects of alleviating obesity, which is representative of glucose metabolism disorders and lipid metabolism disorders, osteoporosis, which is representative of bone homeostasis disorders, and flushing, which is representative of energy metabolism disorders, and thus the extract can be used in food, a medicine and the like so as to prevent, alleviate or treat menopausal disorders.

### TECHNICAL FIELD

The present invention relates to a pharmaceutical composition for preventing or treating menopausal disorders, containing a Tetragonia tetragonoides (Pall.) Kuntze extract. Specifically, the present invention relates to a pharmaceutical composition for preventing or treating menopausal disorders, containing the Tetragonia tetragonoides (Pall.) Kuntze extract or a fraction thereof; a method for preventing or treating menopausal disorders, including a step of administering the pharmaceutical composition; and a food composition containing the extract or a fraction thereof.

### BACKGROUND ART

Up to about 1 year after menopause is called a menopausal transition period, more commonly a menopausal period, and the period is usually 4 to 7 years on average. According to the Korean Society of Menopause, about 89% of Korean women in their 50's is experiencing menopausal symptoms. Due to menopause, disorders of glucose metabolism, lipid metabolism, bone homeostasis, and energy metabolism manifest (Tiano and Mauvais-Jarvis, 2012; Wildman and Sowers, 2011), which can cause menopausal women to develop menopausal disorders, specifically, such as facial flushing, perspiration, symptoms associated with atrophy of the genitourinary system (vaginal dryness, vaginitis due to repeated vaginal infections and urinary tract infections, cystitis, dysuria, and urgent urination), mental instability (concentration disorder and short-term memory disorder, anxiety and nervousness, and decreased memory), variations in skin-joint system (skin dryness and atrophy, myalgia, and arthralgia), increase in fractures due to progression of osteoporosis, and increase in body mass index (BMI).

Administration of synthetic estrogen hormone preparations is known to be capable of resolving some of these disorders, but it is problematic because of side effects that increase risks of breast cancer and endometrial cancer. Accordingly, there have been growing interests in phytoestrogens, which are plant-derived natural estrogens and have similar function and structure to those of estrogens, such as isoflavones, flavonoids, and lignans. Further, with increased evidence that natural compounds such as isoflavonoids act as selective estrogen receptor modulators, studies on natural herbs as candidate substances for preventing, ameliorating, and treating menopausal disorders have been actively conducted. As a result, the product with mixtures of Red Ginseng and natural substances for patients with severe climacteric syndromes (Korean Patent Laid-Open No. 10-2006-0061323) and the like have been developed. However, efficacies thereof remain uncertain (Cano et al., 2008; Somjen et al., 2008).

Thus, there is a desperate need for studies to find new plants containing phytoestrogens that ameliorate menopausal disorders while having the same activity as the above-mentioned modulators in menopausal women. However, the menopausal disorders include multiple disorders of glucose metabolism, lipid metabolism, bone homeostasis, and energy metabolism due to decreased estrogen secretion. Thus, there is a problem that, even in a case where one of these disorders is ameliorated, unless effects of ameliorating the other disorders are exhibited, overall ameliorating effects on the menopausal disorders cannot be expected.

Meanwhile, Tetragonia tetragonoides (Pall.) Kuntze is a perennial grass belonging to the Aizoaceae family, which is referred to as Gaetsangchu or New Zealand spinach. It is also distributed in New Zealand, China, Japan, South Asia, Australia, South America, and the like. In Korea, it is growing on beach sand by forming a community in regions of southern area and Jeju province. From ancient times, in Korea, the Tetragonia tetragonoides (Pall.) Kuntze has been regarded as a precious herb, and is known as one of three great herbs that are good for the stomach, along with an herb ‘root of Atractylodes japonica’ which grows on a mountain and ‘Mallotus japonicas.’ Recently, as efficacies of Tetragonia tetragonoides (Pall.) Kuntze on prophylactic and therapeutic effects of gastrointestinal diseases are known, it is treated in oriental medicines as a precious medicament used for the treatment of gastrointestinal diseases such as cancer, gastritis, gastric ulcer, gastric hyperplasia, and indigestion. Besides, it has been known that a Tetragonia tetragonoides (Pall.) Kuntze extract has an anti-apoptotic activity and thus can be used for the treatment of liver diseases and the like (Korea Patent No. 10-0483183). However, no findings have been reported about effects of the Tetragonia tetragonoides (Pall.) Kuntze on menopausal disorders.

### SUMMARY OF INVENTION

### Technical Problem

Under these circumstances, the present inventors have made extensive efforts to develop a method for ameliorating menopausal disorders using natural herbs. As a result, the present inventors have found that a Tetragonia tetragonoides (Pall.) Kuntze extract is capable of preventing or ameliorating climacteric or menopausal disorders including glucose metabolism disorders, lipid metabolism disorders, bone homeostasis disorders, energy metabolism disorders, and the like. Based on this finding, the present inventors have completed the present invention.

### Solution to Problem

One object of the present invention is to provide a pharmaceutical composition for preventing or treating menopausal disorders, containing a Tetragonia tetragonoides (Pall.) Kuntze extract or a fraction thereof.

Another object of the present invention is to provide a method for preventing or treating menopausal disorders, including a step of administering the pharmaceutical composition to an individual.

Still another object of the present invention is to provide a food composition for preventing or ameliorating menopausal disorders, containing a Tetragonia tetragonoides (Pall.) Kuntze extract or a fraction thereof.

### Advantageous Effects of Invention

The Tetragonia tetragonoides (Pall.) Kuntze extract of the present invention shows effects of ameliorating obesity, which is representative of glucose metabolism disorders and lipid metabolism disorders, osteoporosis, which is representative of bone homeostasis disorders, and flushing, which is representative of energy metabolism disorders, and thus the extract can be used in foods, pharmaceuticals, and the like for preventing, ameliorating, or treating menopausal disorders.

### BRIEF DESCRIPTION OF DRAWINGS

FIG. 1 is a graph showing effects of the Tetragonia tetragonoides (Pall.) Kuntze extract of the present invention on lean body mass in ovariectomized rats (control: ovariectomized rats, positive-control: ovariectomized rats receiving 17β-estradiol).

FIG. 2 is a graph showing effects of the Tetragonia tetragonoides (Pall.) Kuntze extract of the present invention on fat body mass in ovariectomized rats (control: ovariectomized rats, positive-control: ovariectomized rats receiving 17β-estradiol).

FIG. 3 is a graph showing effects of the Tetragonia tetragonoides (Pall.) Kuntze extract of the present invention on serum insulin concentration (AUC of serum insulin) according to results of oral glucose tolerance test in ovariectomized rats (control: ovariectomized rats, positive-control: ovariectomized rats receiving 17β-estradiol). Specifically, it shows an insulin-secreting ability in a case where blood glucose increases, and shows a value obtained by calculating areas of a curve of changes of blood glucose for 0 to 50 minutes and 50 to 120 minutes in measurement results of serum insulin. An average of a total area under curve on serum insulin was calculated in a trapezoidal manner.

FIG. 4 is a graph showing results of insulin tolerance test in ovariectomized rats, which are used as an index indicating insulin resistance. This shows effects of the Tetragonia tetragonoides (Pall.) Kuntze extract of the present invention on serum glucose levels in a case where insulin was injected by intraperitoneal way (control: ovariectomized rats, positive-control: ovariectomized rats receiving 17β-estradiol). Specifically, insulin (0.75 U/kg body weight) was intraperitoneally injected into rats fasted for 5 hours, and then measurement was performed every 15 minutes for 90 minutes.

FIG. 5 is a graph showing effects of the Tetragonia tetragonoides (Pall.) Kuntze extract of the present invention on bone mineral density in ovariectomized rats (control: ovariectomized rats, positive-control: ovariectomized rats receiving 17β-estradiol). All values are expressed as means±SD. A statistical significance of each result was confirmed by the Tukey's test, and different subscripts represented by a and b in the same row indicate that a significant difference exists at p<0.05.

FIG. 6 is a graph showing the effect of the Tetragonia tetragonoides (Pall.) Kuntze extract of the present invention on tail skin temperature in ovariectomized rats (Control: ovariectomized rats, positive-control: ovariectomy Rat receiving 17β-estradiol). All values are expressed as means±SD. A statistical significance of each result was confirmed by the Tukey's test, and different subscripts represented by a and b in the same row indicate that a significant difference exists at p<0.05.

### DESCRIPTION OF EMBODIMENTS

In order to achieve the above objects, in one aspect, there is provided a pharmaceutical composition for preventing or treating menopausal disorders, containing a Tetragonia tetragonoides (Pall.) Kuntze extract or a fraction thereof.

The Tetragonia tetragonoides (Pall.) Kuntze extract of the present invention exhibits effects of decreasing visceral fat, subcutaneous fat, serum glucose levels, insulin levels, HOMA-IR concentration, triglyceride levels, and total cholesterol; increasing HDL cholesterol, daily energy consumption, and fat oxidation; increasing bone mineral density; or decreasing skin temperature, and thus can be used for preventing, treating, or ameliorating menopausal disorders showing various symptoms such as osteoporosis, obesity, and flushing.

As used herein, the term “Tetragonia tetragonoides (Pall.) Kuntze” refers to a perennial grass belonging to the Aizoaceae family. This grass is 40 to 60 cm high, has no hair but with wart-like protrusions, and stands obliquely and extends sideways due to many branches coming from below. In Korea, it is known that Tetragonia tetragonoides (Pall.) Kuntze is growing on beach sand by forming a community in regions of southern area and Jeju province. Effects of the Tetragonia tetragonoides (Pall.) Kuntze for ameliorating menopausal disorders have not been known at all so far and were first identified by the present inventors. In the present invention, the Tetragonia tetragonoides (Pall.) Kuntze can be purchased from commercial sources, or can be used as one collected or cultivated from nature.

As used herein, the term “extract” includes an extract liquid itself and extracts of all formulations which can be formed using the extract liquid, such as an extract liquid obtained by extracting Tetragonia tetragonoides (Pall.) Kuntze, a diluted solution or concentrate of the extract liquid, a dried product obtained by drying the extract liquid, a crude-purified or purified product of the extract liquid, or mixtures thereof.

A method for extracting Tetragonia tetragonoides (Pall.) Kuntze is not particularly limited, and extraction can be performed according to methods commonly used in the art. Non-limiting examples of the extraction method include hydrothermal extraction method, ultrasonic extraction method, filtration method, and reflux extraction method. These methods may be carried out alone or in combination of two or more thereof.

In the present invention, a type of an extraction solvent used for extracting the Tetragonia tetragonoides (Pall.) Kuntze is not particularly limited, and any solvent known in the art can be used. Non-limiting examples of the extraction solvent include water, alcohol, or a mixed solvent thereof. These solvents may be used alone or in combination of one or more thereof. As a specific example thereof, water can be used, but the solvent is not limited thereto. In a case where the alcohol is used as a solvent, specifically, an alcohol having 1 to 4 carbon atoms can be used.

As used herein, the term “fraction” refers to a resulting product obtained by performing fractionation to separate a specific component or a group of specific components from a mixture containing various different constituent components.

A fractionation method for obtaining the fraction in the present invention is not particularly limited, and fractionation can be performed according to methods commonly used in the art. Non-limiting examples of the fractionation method include a method of obtaining a fraction from an extract of the present invention, which has been obtained by extracting Tetragonia tetragonoides (Pall.) Kuntze, by treating the extract with a predetermined solvent.

A type of the fractionation solvent used for obtaining the fraction in the present invention is not particularly limited, and any solvent known in the art can be used. Non-limiting examples of the fractionation solvent include polar solvents such as water and alcohol; and non-polar solvents such as hexane, ethyl acetate, chloroform, and dichloromethane. These may be used alone or in combination of one or more thereof.

Further, the extract or fraction may be prepared and used in the form of a dry powder after extraction, but the present invention is not limited thereto.

As used herein, the term “menopausal disorders” refers to diseases that manifest various abnormal symptoms occurring during a menopausal period, which is a period when menstruation is stopped. During the period, in general, ovarian function deteriorates due to aging and female hormones are secreted in a small amount, which results in hormone imbalance in a body. The menopausal disorders manifest symptoms resulting from decreased estrogen secretion, and such symptoms include, but are not limited to, one or more symptoms selected from the group consisting of glucose metabolism disorders, lipid metabolism disorders, bone homeostasis disorders, and energy metabolism disorders. As specific examples thereof, symptoms which are representative of the glucose metabolism disorders and the lipid metabolism disorders include obesity; symptoms which are representative of the bone homeostasis disorders include osteoporosis; and symptoms which are representative of the energy metabolism disorders include flushing, but the symptoms are not limited thereto. More specific examples of the menopausal disorders may be osteoporosis, flushing, or a combination thereof.

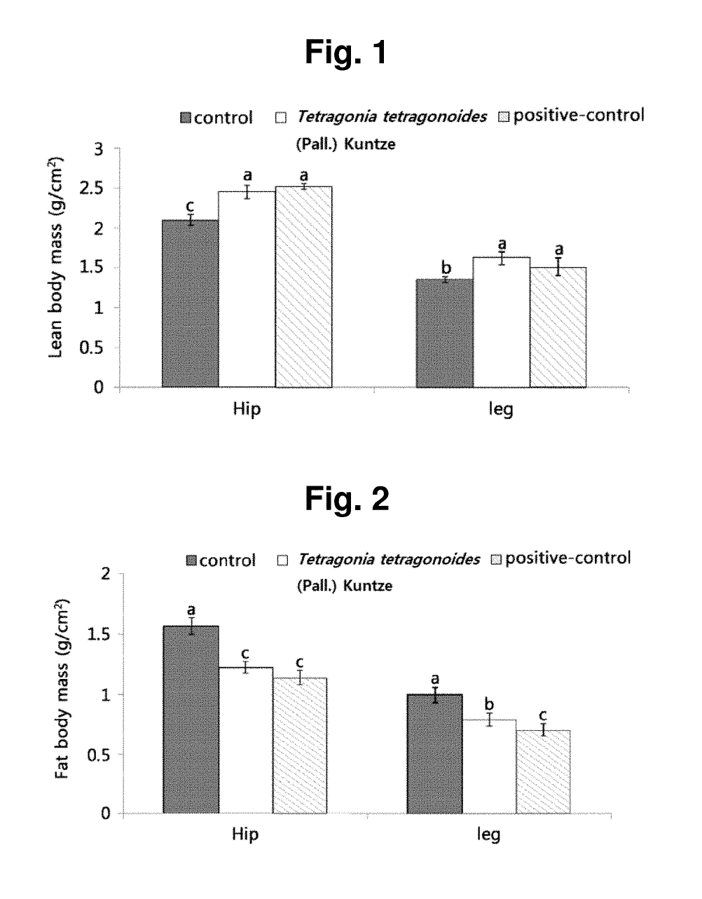

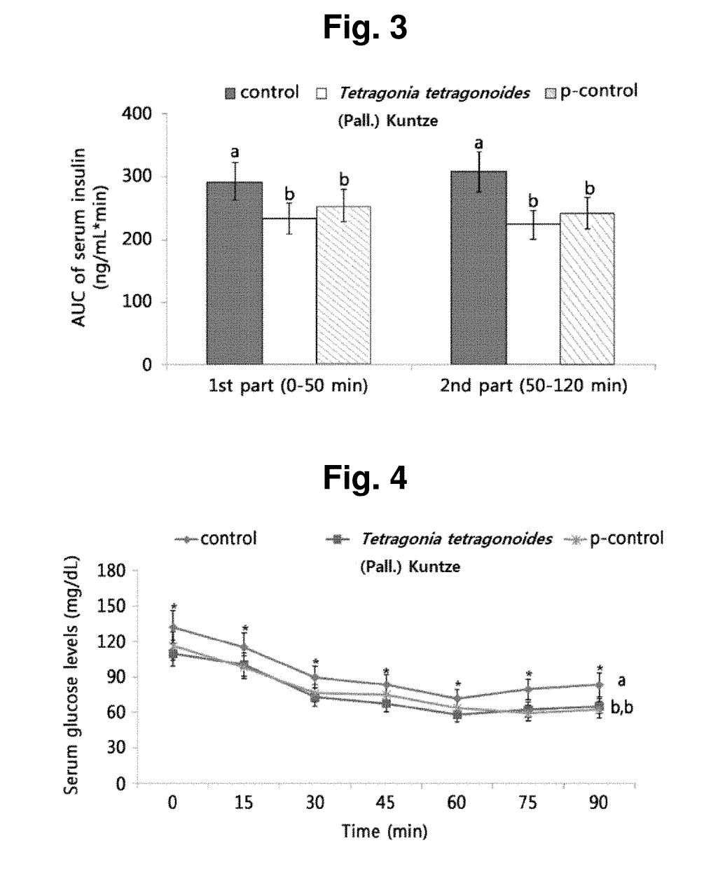

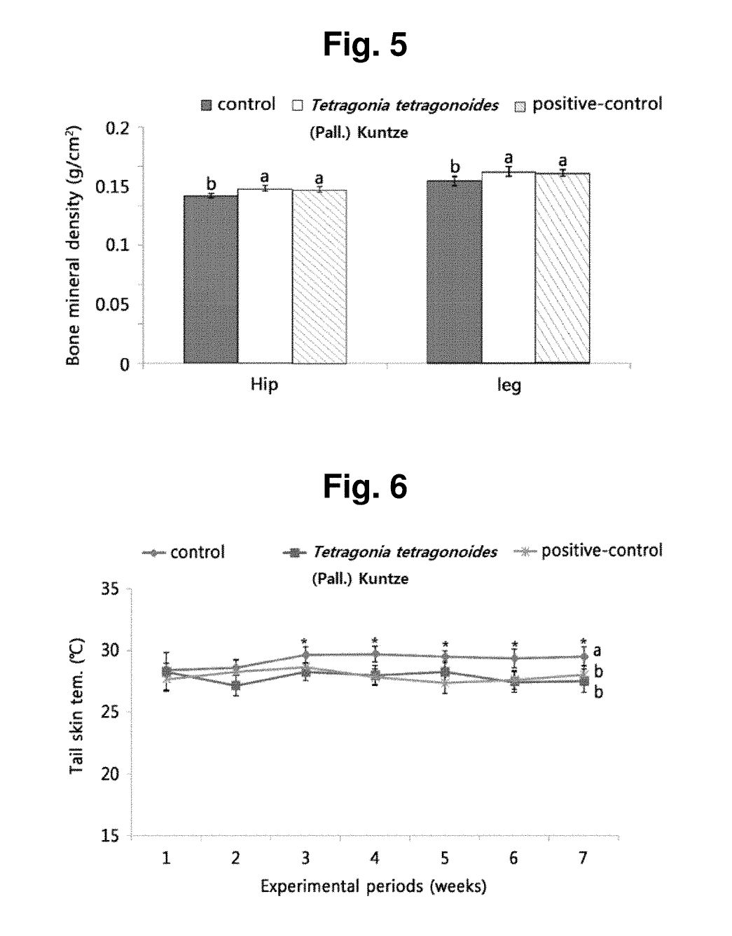

In a specific embodiment of the present invention, rats had been ovariectomized to induce menopause and were fed with the Tetragonia tetragonoides (Pall.) Kuntze extract of the present invention. As a result, the following facts were confirmed: a mass of visceral fat including peri-uterine fat and fat in retroperitoneum, and subcutaneous fat in hip and leg portions were decreased (Table 1, and FIGS. 1 to 2); daily energy consumption and fat oxidation were increased (Table 1); serum glucose levels, insulin levels, HOMA-IR concentration, triglyceride levels, and total cholesterol were decreased, but HDL cholesterol was increased (Table 2, FIGS. 3 to 4); bone mineral density was increased (FIG. 5); and skin temperature was decreased (FIG. 6). This suggests that the Tetragonia tetragonoides (Pall.) Kuntze extract can be useful for preventing, treating, or ameliorating obesity, osteoporosis, or flushing which is a representative abnormal menopausal symptom.

As used herein, the term “prevention” refers to any action that inhibits or delays menopausal disorders by administration of a pharmaceutical composition containing the Tetragonia tetragonoides (Pall.) Kuntze extract of the present invention or a fraction thereof.

As used herein, the term “treatment” refers to any action that improves or beneficially modifies menopausal disorders by administration of the pharmaceutical composition.

The pharmaceutical composition of the present invention may contain the Tetragonia tetragonoides (Pall.) Kuntze extract or a fraction thereof in an amount of 0.0001 to 50% by weight, specifically 0.01 to 10% by weight, with respect to a weight of the total composition, but the amount is not limited thereto.

The pharmaceutical composition of the present invention may further contain a pharmaceutically acceptable carrier, excipient, or diluent commonly used in the preparation of a pharmaceutical composition, in which the carrier may include a non-naturally occurring carrier.

As used herein, the term “pharmaceutically acceptable” means that properties with no toxicity to cells or humans exposed to the composition are exhibited.

Specifically, the pharmaceutical composition may be formulated in the form of oral formulations such as powders, granules, tablets, capsules, suspensions, emulsions, syrups, and aerosols, external preparations, suppositories, and sterile injectable solutions, respectively, according to commonly used methods, and used. In the present invention, the carrier, excipient, and diluent which may be contained in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinyl pyrrolidone, water, methylhydroxybenzoate, propylhydroxybenzoate, talc, magnesium stearate, and mineral oil. In a case of making preparations, a commonly used diluent or excipient such as a filler, an extender, a binder, a wetting agent, a disintegrant, and a surfactant is used to make preparations. Solid preparations for oral administration include tablets, pills, powders, granules, capsules, and the like, which are prepared by admixture with at least one excipient, for example, starch, calcium carbonate, sucrose or lactose, and gelatin. In addition, besides simple excipients, a lubricant such as magnesium stearate and talc is also used. Liquid preparations for oral use include suspensions, oral liquids, emulsions, syrups, and the like, and may contain various excipients, for example, wetting agents, sweeteners, fragrances, and preservatives, in addition to water or liquid paraffin which is a commonly used simple diluent. Preparations for parenteral administration include sterile aqueous solutions, non-aqueous solutions, suspensions, emulsions, freeze-dried preparations, and suppositories. As a non-aqueous solvent or suspending agent, propylene glycol, polyethylene glycol, vegetable oil such as olive oil, injectable ester such as ethyl oleate, and the like can be used. As a base of suppositories, witepsol, macrogol, tween 61, cacao butter, laurin butter, glycerogelatin, and the like can be used.

In another aspect, there is provided a method for preventing or treating menopausal disorders, including a step of administering the pharmaceutical composition to an individual having or at risk of developing menopausal disorders.

In this case, definitions for the Tetragonia tetragonoides (Pall.) Kuntze, extract, fraction, menopausal disorders, prevention, and treatment are as described above.

As used herein, the term “administration” refers to introducing a predetermined substance into an individual in an appropriate manner.

As used herein, the term “individual” refers to all animals such as rats, mice, and livestock, including humans, who have or are at risk of developing menopausal disorders. A specific example thereof may be a mammal including a human.

The method for preventing or treating menopausal disorders according to the present invention may, specifically, include a step of administering, to an individual, a pharmaceutically effective amount of a pharmaceutical composition for preventing or treating menopausal disorders, containing a Tetragonia tetragonoides (Pall.) Kuntze extract or a fraction thereof.

As used herein, the term “pharmaceutically effective amount” refers to an amount sufficient to treat diseases at a reasonable benefit/risk ratio applicable to medical treatment and not causing side effects. An effective dosage level may be readily determined by those skilled in the art depending on factors, including a patient's sex, age, body weight, health condition, type of disease and severity thereof, activity of drug, sensitivity to drug, mode of administration, administration time, route of administration, excretion rate, duration of treatment, and drugs used in combination or concurrently, and other factors well known in the medical field.

Specifically, the composition of the present invention may be administered at a dose of 0.0001 to 100 mg/kg body weight per day, and more specifically 0.001 to 100 mg/kg body weight, based on a solid content. Administration may be carried out in such a way that the recommended dose may be administered once a day or divided into several doses.

In the method for preventing or treating menopausal disorders according to the present invention, the route of administration and the mode of administration for administering the composition are not particularly limited, and any route of administration and any mode of administration may be followed as long as the composition can reach a desired target site. Specifically, the composition may be administered via various routes including oral or parenteral routes. Non-limiting examples of the route of administration include administration via oral, rectal, topical, intravenous, intraperitoneal, intramuscular, intraarterial, transdermal, or intranasal route, or through inhalation.

In still another aspect, there is provided a food composition for preventing or ameliorating menopausal disorders, containing a Tetragonia tetragonoides (Pall.) Kuntze extract or a fraction thereof.

In this case, definitions for the Tetragonia tetragonoides (Pall.) Kuntze, extract, fraction, menopausal disorders, and prevention are as described above.

As used herein, the term “amelioration” refers to any action that at least decreases parameters, such as a degree of symptom, associated with a condition to be treated by administration of the composition containing the Tetragonia tetragonoides (Pall.) Kuntze extract of the present invention or a fraction thereof.

As used herein, the term “food” is intended to include meat, sausages, bread, chocolate, candies, snacks, confectioneries, pizza, ramen, other noodles, gums, dairy products including ice cream, various soups, beverages, teas, drinks, alcoholic beverages, vitamin complexes, functional food, health food, and the like, and includes all foods in a common sense.

The food composition of the present invention can be ingested on a daily basis, which makes it possible to expect great ameliorating effects on menopausal disorders. Thus, the food composition of the present invention can be very useful for health promotion purposes.

The functional food is synonymous with a food for special health use (FoSHU) and refers to a food which has been processed so that, in addition to nutritional supplementation, biological regulation functions are efficiently exerted, and which has high medical or medicinal effects. Here, ‘function (or functional)’ refers to regulating nutrients with respect to structures and functions of a human body or obtaining beneficial effects, such as physiological actions, which are useful for health use. The food of the present invention can be prepared by methods commonly used in the art and can be prepared by adding raw materials and components which are commonly added in the art at the time of preparing the same. In addition, the food can also be prepared in any formulation without limitations as long as the formulation is acceptable as a food. The food composition of the present invention can be prepared in various types of formulations. Unlike general medicines, due to use of foods as raw materials, the food composition of the present invention has an advantage that, for example, there are no side effects which may be caused by a long-term intake of a medicine, and has excellent portability. Thus, the food of the present invention can be ingested as a supplement to enhance effects of ameliorating menopausal disorders.

The health food refers to a food having an active health maintenance or promotion effects as compared with a general food, and a health supplement food refers to a food for health supplement purposes. The terms functional food, health food, and health supplement food are interchangeably used, as the case may be.

Specifically, the functional food refers to a food that is prepared by adding the compound of the present invention to food material such as beverages, teas, spices, gums, or confectioneries, or by performing encapsulation, pulverization, suspending, or the like, and that produces certain health effects in a case of being ingested. Unlike general medicines, due to use of foods as raw materials, such functional food has an advantage that there are no side effects which may be caused by a long-term intake of a medicine.

The food composition may further contain a physiologically acceptable carrier. A type of the carrier is not particularly limited, and any carrier can be used as long as it is commonly used in the art.

Further, the food composition may contain additional components that can be commonly used in food compositions to improve odor, taste, visual appearance, and the like. For example, vitamins A, C, D, E, B1, B2, B6, B12, niacin, biotin, folate, pantothenic acid, and the like may be contained. In addition, minerals such as zinc (Zn), iron (Fe), calcium (Ca), magnesium (Mg), manganese (Mn), copper (Cu), and chromium (Cr); and amino acids such as lysine, tryptophan, cysteine, and valine may be contained.

Further, the food composition may contain food additives such as preservatives (potassium sorbate, sodium benzoate, salicylic acid, sodium dehydroacetate, and the like), disinfectants (bleaching powder, high grade bleaching powder, sodium hypochlorite, and the like), antioxidants (butylhydroxyanisole (BHA), butyl hydroxytoluene (BHT), and the like), coloring agents (tar color and the like), color formers (sodium nitrite and the like), bleaching agents (sodium sulfite), seasonings (sodium glutamate including MSG, and the like), sweeteners (Dulcin, cyclamate, saccharin, sodium, and the like), flavoring agents (vanillin, lactones, and the like), blowing agents (alum, potassium D-bitartrate, and the like), fortifying agents, emulsifying agents, thickeners (thickening agent), coating agents, gum bases, antifoaming agents, solvents, and modifying agents. The additives may be selected depending on a type of food and used in appropriate amounts.

As an example of the food composition of the present invention, it can be used as a health beverage composition. In this case, various flavorings, natural carbohydrates, or the like may be contained as additional components as in ordinary beverages. The above-mentioned natural carbohydrates may be monosaccharides such as glucose and fructose; disaccharides such as maltose and sucrose; polysaccharides such as dextrin and cyclodextrin; and sugar alcohols such as xylitol, sorbitol, and erythritol. As sweeteners, natural sweeteners such as Thaumatin and extracts of Stevia; synthetic sweeteners such as saccharin and aspartame; and the like can be used. A proportion of the natural carbohydrate may be generally about 0.01 to 0.04 g, and specifically about 0.02 to 0.03 g, per 100 mL of the health beverage composition of the present invention.

In addition to the above, the health beverage composition may contain various nutrients, vitamins, electrolytes, flavors, coloring agents, pectic acids, salts of pectic acids, alginic acids, salts of alginic acids, organic acids, protective colloidal thickeners, pH adjusting agents, stabilizers, preservatives, glycerin, alcohols, carbonating agents, or the like. Besides, it may contain flesh for the production of natural fruit juices, fruit juice beverages, or vegetable beverages. These components can be used independently or in combination. A proportion of such additives is not very important, and is generally selected in a range of 0.01 to 0.1 parts by weight per 100 parts by weight of the health beverage composition of the present invention.

Hereinafter, constitution and effects of the present invention will be described in more detail by way of examples. These examples are only for illustrating the present invention, and a scope of the present invention is not limited by these examples.

### Preparation Example 1. Preparation of Tetragonia tetragonoides (Pall.) Kuntze Extract

In order to prepare a Tetragonia tetragonoides (Pall.) Kuntze extract, a Tetragonia tetragonoides (Pall.) Kuntze collected from Jeju Island in Korea was used, water or ethanol in an amount 10 times larger than the Tetragonia tetragonoides (Pall.) Kuntze specimen was added, and then treatment at 70° C. for 12 hours was carried out to obtain a Tetragonia tetragonoides (Pall.) Kuntze extract. The Tetragonia tetragonoides (Pall.) Kuntze extract was filtered with a 0.4 μm filter, and then concentrated and freeze-dried using a rotary evaporator to prepare a Tetragonia tetragonoides (Pall.) Kuntze extract.

### Experimental Example 1. Construction of Menopause-Induced Animal Model

In order to confirm efficacies of the Tetragonia tetragonoides (Pall.) Kuntze extract prepared according to Preparation Example 1 for ameliorating menopausal disorders, a menopause-induced mouse model was constructed.

Rats were ovariectomized to induce menopause, and weight of uterus and serum levels of 17β-estradiol that is a kind of female hormones were measured to determine whether menopause was induced in the rats. Specifically, the rats were sacrificed, and then their uteri were removed and weighed. A serum concentration of 17β-estradiol was measured with an RIA kit (Linco Research Inc.). Results measured according to the above are summarized in Table 1 below.

TABLE 1
Evaluation on menopause inducement in ovariectomized rats

| | Normal | | Tetragonia | |
|---|---|---|---|---|
| | control | | tetragonoides | |
| | with ovary | Control | (Pall.) Kuntze | Positive-control |
| | (n = 12) | (n = 12) | (n = 12) | (n = 12) |
| Weight of | 0.68 | 0.23 ± 0.07^{b} | 0.23 ± 0.07^{b} | 0.61 ± 0.22^{a} |
| uterus (g) | | | | |
| Serum levels | 6.2 ± 0.9 | 1.7 ± 0.5^{b} | 1.8 ± 0.6^{b} | 7.5 ± 1.2^{a} |
| of 17β- | | | | |
| estradiol | | | | |
| (pg/ml) | | | | |

(All values are expressed as means ± SD. A statistical significance of each result was confirmed by the Tukey's test, and different subscripts represented by a and b in the same row indicate that a significant difference exists at p < 0.05.)

As a result, as shown in Table 1, it was confirmed that as compared with normal control rats with ovary, control ovariectomized rats not fed with a Tetragonia tetragonoides (Pall.) Kuntze extract and ovariectomized rats fed with the extract showed a significant decrease in weight of uterus or serum levels of 17β-estradiol, whereas it was confirmed that positive-control ovariectomized rats receiving the hormone showed similar weight of uterus or levels of the hormone to the normal control rats.

The results could confirm that menopause was well induced in the ovariectomized rats.

### Example 1. Confirmation of Efficacies of Tetragonia tetragonoides (Pall.) Kuntze Extract for Ameliorating Obesity

### Example 1-1. Confirmation of Efficacies on Visceral Fat

In order to confirm efficacies of the Tetragonia tetragonoides (Pall.) Kuntze extract prepared according to Preparation Example 1 for ameliorating menopausal obesity, rats were ovariectomized and, at the same time, obesity-induced. The rats were fed with the Tetragonia tetragonoides (Pall.) Kuntze extract, and effects of the extract on visceral fat were evaluated.

Specifically, the ovariectomized rats were fed with a high-fat diet (HFD) consisting of 40% energy (En %) of carbohydrate, 20 En % of protein, and 40 En % of fat, along with dextrin. The high-fat diet was made to contain 2% (w/w) of the Tetragonia tetragonoides (Pall.) Kuntze extract, and the rats were offered ad libitum access to the diet for 8 weeks. On the other hand, in the positive-control, the ovariectomized rats received 17β-estradiol which is a hormone preparation and were fed with a diet in which the high fat diet is mixed with dextrin for 8 weeks. Then, at the end of the experiment, a body weight, a mass of visceral fat including peri-uterine fat and fat in retroperitoneum, an energy consumption, and the like were measured.

The energy consumption was analyzed by indirect calorimetry. Specifically, after the Tetragonia tetragonoides (Pall.) Kuntze extract was fed for 8 weeks, and a fasting state of 6 hours was induced, the energy consumption was measured at the time of beginning of a dark period in light/dark cycle. In order to analyze calorimetric parameters, a metabolic chamber (airflow=800 ml/min) equipped with a computer-controlled O₂ and CO₂ measurement system (BIOPAC Systems, Inc., Goleta, Calif.) was utilized, and respiratory quotient (RQ) and resting energy expenditure (REE) were calculated using equations. An average oxygen uptake (VO₂) and an average carbon dioxide emission (VCO₂) were measured over 30 minutes. After the experiment, data were averaged at 1 minute intervals and the VO₂ and VCO₂ values were calibrated to a body size (kg^{0.75}). Oxidation of carbohydrate and fat was calculated as a non-protein oxygen uptake, that is, a relative oxidation rate and an oxygen amount consumed per gram (g) of oxidized substrate.

Results measured according to the above are summarized in Table 2 below.

TABLE 2
Metabolic parameters at end of experiment

| | | Tetragonia | |
|---|---|---|---|
| | | tetragonoides | Positive- |
| | Control | (Pall.) | control |
| | (n = 12) | Kuntze (n = 12) | (n = 12) |
| Body weight (g) | 404 ± 27^{a} | 389 ± 20^{a} | 369 ± 26^{b} |
| Body weight gain (g) | 99.6 ± 14.2^{a} | 83.2 ± 12.3^{b} | 70.0 ± 11.5^{c} |
| Peri-uterine fat (g) | 15.1 ± 2.6^{a} | 11.3 ± 2.1^{c} | 7.2 ± 1.6^{d} |
| Fat in retroperitoneum | 9.9 ± 1.8^{a} | 6.4 ± 2.7^{b} | 5.7 ± 1.6^{c} |
| (g) | | | |
| Visceral fat (g) | 25.0 ± 4.1^{a} | 17.7 ± 3.8^{b} | 12.9 ± 2.6^{c} |
| Weight of uterus (g) | 0.23 ± 0.07^{b} | 0.23 ± 0.07^{b} | 0.61 ± 0.22^{a} |
| Calorie uptake | 12.9 ± 2.1 | 15.4 ± 4.4 | 13.9 ± 3.3 |
| (Kcal/day) | | | |
| Daily energy | 101 ± 13^{b} | 118 ± 14^{a} | 117 ± 15^{a} |
| consumption | | | |
| (kcal/kg^{0.75}/day) | | | |
| Carbohydrate oxidation | 6.5 ± 0.8^{a} | 4.4 ± 0.6^{c} | 5.6 ± 0.7^{b} |
| (mg/kg^{0.75}/min) | | | |
| Fat oxidation | 4.2 ± 0.7^{d} | 8.1 ± 1.2^{a} | 6.9 ± 0.8^{b} |
| (mg/kg^{0.75}/min) | | | |

(All values are expressed as means ± SD. A statistical significance of each result was confirmed by the Tukey's test, and different subscripts represented by a, b, c, and d in the same row indicate that a significant difference exists at p < 0.05.)

As a result, as shown in Table 2, it was confirmed that as compared with the control ovariectomized rats that were not fed with the Tetragonia tetragonoides (Pall.) Kuntze extract, the ovariectomized rats fed with the extract showed decrease in body weight gain, peri-uterine fat, fat in retroperitoneum, and visceral fat.

Besides, it was confirmed that the ovariectomized rats fed with the extract showed higher daily energy consumption, in particular, higher fat oxidation, than the control ovariectomized rats as well as the positive-control rats that were ovariectomized and received 17β-estradiol. Subsequently, it was confirmed that the positive-control rats receiving 17β-estradiol showed increase in weight of uterus, while the rats receiving the Tetragonia tetragonoides (Pall.) Kuntze extract showed no increase in weight of uterus. The increase in weight of uterus is one of typical side effects in hormone therapies, meaning that proliferation of uterus has occurred. From the results, it can be seen that the Tetragonia tetragonoides (Pall.) Kuntze extract exhibits effects of ameliorating or treating desired menopausal disorders without side effects.

Further, as shown in FIGS. 1 and 2, it was confirmed that the ovariectomized rats fed with the extract showed increase in weight excluding fat in hip and leg portions, while showing decrease in weight of fat. Thus, it was confirmed that the Tetragonia tetragonoides (Pall.) Kuntze extract has effects of decreasing visceral fat as well as subcutaneous fat.

### Examples 1-2. Confirmation of Efficacies on Blood

In order to confirm efficacies of the Tetragonia tetragonoides (Pall.) Kuntze extract prepared according to Preparation Example 1 for ameliorating menopausal obesity, rats were ovariectomized and, at the same time, obesity-induced. The rats were fed with the Tetragonia tetragonoides (Pall.) Kuntze extract, and effects of the extract on obesity-related parameters in blood were evaluated.

The ovariectomized rats were fed with the Tetragonia tetragonoides (Pall.) Kuntze by the method according to Example 1-1. Then, at the end of the experiment, the rats were fasted overnight and masses of serum glucose, insulin, and triglyceride were measured. Results are summarized in Table 3 below. Specifically, the rats were fasted for 16 hours, and then blood was collected from tails. Fasting serum glucose levels were measured by a Beckman glucometer and serum insulin concentrations were measured by radioimmunoassay. Fasting blood glucose, food and water intake, and body weight were measured at 10 a.m. on every Tuesday. Based on the above results, a homeostasis model assessment of insulin resistance (HOMA-IR) concentration which is used as a marker of insulin resistance was calculated, and the following Equation 1 was used for such calculation.

HOMA-IR=fasting insulin(μIU/ml)×fasting glucose(mM)/22.5 [Equation 1]

TABLE 3
Serum triglyceride, glucose, and insulin levels in overnight-fasted rats

| | | Tetragonia | |
|---|---|---|---|
| | | tetragonoides | Positive- |
| | Control | (Pall.) | control |
| | (n = 12) | Kuntze (n = 12) | (n = 12) |
| Glucose levels | 129 ± 14^{a} | 108 ± 12^{b} | 109 ± 13^{b} |
| (mg/dL) | | | |
| Insulin levels | 1.45 ± 0.25^{a} | 0.88 ± 0.16^{c} | 1.14 ± 0.19^{b} |
| (ng/mL) | | | |
| HOMA-IR | 10.4 ± 1.5^{a} | 5.3 ± 0.8^{d} | 6.8 ± 0.9^{c} |
| Triglyceride levels | 73.8 ± 6.8^{a} | 63.1 ± 5.3^{b} | 65.7 ± 5.8^{b} |
| (mg/dL) | | | |
| Total cholesterol | 103.5 ± 8.4^{a} | 95.3 ± 7.8^{b} | 85.0 ± 7.6^{c} |
| (mg/dL) | | | |
| HDL cholesterol | 19.4 ± 1.2^{b} | 22.1 ± 1.7^{a} | 23.2 ± 1.5^{a} |
| (mg/dL) | | | |

(All values are expressed as means ± SD. A statistical significance of each result was confirmed by the Tukey's test, and different subscripts represented by a, b, c, and d in the same row indicate that a significant difference exists at p < 0.05.)

As a result, as shown in Table 3, and FIGS. 3 and 4, it was confirmed that as compared with the control ovariectomized rats that were not fed with the Tetragonia tetragonoides (Pall.) Kuntze extract, the ovariectomized rats fed with the extract showed decrease in all of serum glucose levels, insulin levels, HOMA-IR concentration, triglyceride levels, and total cholesterol, while showing increase in HDL cholesterol. Besides, it was confirmed that the Tetragonia tetragonoides (Pall.) Kuntze extract showed superior or similar effects to the positive-control rats receiving 17β-estradiol in all of the above-mentioned items.

### Example 2. Confirmation of Efficacies of Tetragonia tetragonoides (Pall.) Kuntze Extract for Ameliorating Osteoporosis

In order to confirm efficacies of the Tetragonia tetragonoides (Pall.) Kuntze extract prepared according to Preparation Example 1 for ameliorating menopausal osteoporosis, ovariectomized rats were fed with the Tetragonia tetragonoides (Pall.) Kuntze extract, and effects of the extract on bone mineral density were evaluated.

The ovariectomized rats were fed with the Tetragonia tetragonoides (Pall.) Kuntze by the method according to Example 1-1. Then, at the end of the experiment, bone mineral density of the rats was measured. Specifically, the rats were anesthetized using ketamine (100 mg/kg body weight) and xylazine (10 mg/kg body weight), laid prone, and then hind legs thereof were maintained in an external rotation state using a sticky tape. The hip, knee, and ankle joints were bent 90°. Using an absorptiometer (pDEXA Sabre; Norland Medical Systems Inc., Fort Atkinson, Wis., USA) equipped with a software suitable for measuring bone mineral density in small animals, bone mineral density was measured through dual-energy X-ray absorptiometry (DEXA) in the right femur and lumbar spine on the 11th week after the experiment. In a similar manner, abdominal fat and lean mass were measured through the dual-energy X-ray absorptiometry (DEXA). The absorptiometer was calibrated daily using phantoms supplied by the manufacturer.

As a result, as shown in FIG. 5, it was confirmed that as compared with the control ovariectomized rats that were not fed with the Tetragonia tetragonoides (Pall.) Kuntze extract, the ovariectomized rats fed with the extract showed increase in bone mineral density of hip and leg. Besides, it was confirmed that the Tetragonia tetragonoides (Pall.) Kuntze extract showed superior or similar effects to the positive-control rats receiving 17β-estradiol with respect to effects of increasing bone mineral density.

### Example 3. Confirmation of Efficacies of Tetragonia tetragonoides (Pall.) Kuntze Extract for Ameliorating Flushing

In order to confirm efficacies of the Tetragonia tetragonoides (Pall.) Kuntze extract prepared according to Preparation Example 1 for ameliorating menopausal flushing, ovariectomized rats were fed with the Tetragonia tetragonoides (Pall.) Kuntze extract, and effects of the extract on increase in skin temperature accompanied by flushing induction were evaluated.

The ovariectomized rats were fed with the Tetragonia tetragonoides (Pall.) Kuntze by the method according to Example 1-1. Then, at the end of the experiment, tail skin temperature in the rats was measured. Specifically, before measuring weight at 10:00 on every Tuesday, tail skin temperature was measured three times using an infrared thermometer and a mean value thereof was used.

As a result, as shown in FIG. 6, it was confirmed that as compared with the control ovariectomized rats that were not fed with the Tetragonia tetragonoides (Pall.) Kuntze extract, the ovariectomized rats fed with the extract showed decrease in tail skin temperature. Besides, it was confirmed that the Tetragonia tetragonoides (Pall.) Kuntze extract showed superior or similar effects to the positive-control rats receiving 17β-estradiol with respect to decrease in skin temperature.

From the above description, those skilled in the art will be able to understand that the present invention may be implemented in other specific modes without changing a technical spirit or an essential feature thereof. In this regard, it should be understood that the above-described examples are illustrative in all respects and not restrictive. Regarding a scope of the present invention, it should be construed that all of changed or modified forms derived from meaning and scope of the claims as described later and an equivalent concept thereto, rather than the above detailed description, are included in the scope of the present invention.

1. A pharmaceutical composition for preventing or treating menopausal disorders, comprising a Tetragonia tetragonoides (Pall.) Kuntze extract or a fraction thereof.

2. The pharmaceutical composition according to claim 1,
wherein the Tetragonia tetragonoides (Pall.) Kuntze extract is obtained by performing extraction with at least one solvent selected from the group consisting of water, an alcohol having 1 to 4 carbon atoms, and a mixed solvent thereof.

3. The pharmaceutical composition according to claim 1,
wherein the menopausal disorders include symptoms due to decreased estrogen secretion.

4. The pharmaceutical composition according to claim 3,
wherein the symptoms include at least one symptom selected from the group consisting of glucose metabolism disorders, lipid metabolism disorders, bone homeostasis disorders, and energy metabolism disorders.

5. The pharmaceutical composition according to claim 1,
wherein the menopausal disorders are osteoporosis, flushing, or a combination thereof.

6. The pharmaceutical composition according to claim 1,
wherein the composition further comprises a pharmaceutically acceptable carrier, excipient, or diluent.

7. A method for preventing or treating menopausal disorders, comprising a step of administering the composition according to claim 1 to a non-human individual having or at risk of developing menopausal disorders.

8. A food composition for preventing or ameliorating menopausal disorders, comprising a Tetragonia tetragonoides (Pall.) Kuntze extract or a fraction thereof.
